(19) United States
(12) Patent Application Publication
(10) Pub. No.: US 2022/0362131 A1
(43) Pub. Date: Nov. 17, 2022

(54) COMPOSITION COMPRISING AMPS POLYMER AND POLYSACCHARIDE

(71) Applicant: L'OREAL, PARIS (FR)

(72) Inventors: Yukiho TAKATA, Kanagawa (JP); Mitsuru SHIMATANI, Kanagawa (JP); Kung Chi HSU, Kanagawa (JP); Mutsue HASHIMOTO, Kanagawa (JP)

(21) Appl. No.: 17/753,103

(22) PCT Filed: Aug. 7, 2020
(86) PCT No.: PCT/JP2020/030926

§ 371 Date: Feb. 18, 2022

(30) Foreign Application Priority Data
Aug. 28, 2019 (JP) 2019-155878

(51) Int. Cl. A61K 8/81; A61K 8/06; A61K 8/73; A61K 8/368; A61K 8/34; A61K 8/99; A61K 8/9728; A61Q 19/00

(52) U.S. Cl. CPC A61K 8/8188; A61K 8/062; A61K 8/73; A61K 8/368; A61K 8/345; A61K 8/99; A61K 8/9728; A61Q 19/00

(57) ABSTRACT

The present invention relates to a two-phase composition comprising an oil phase comprising (a) at least one oil, and an aqueous phase comprising: (b) at least one crosslinked or non-crosslinked acrylamido-2-methylpropanesulfonic acid (AMPS) homopolymer; (c) at least one polysaccharide derived from microorganisms; and (d) water. The composition according to the present invention has two visually distinct phases, can transform into a single phase composition when being mixed, and can maintain the single phase for a long period of time without mixing.

### TECHNICAL FIELD

The present invention relates to a two-phase or bi-phase composition which has two visually distinct phases and is capable of transforming into a single phase composition that cannot transform again into a two-phase or bi-phase composition for a long period of time.

### BACKGROUND ART

A two-phase or bi-phase cosmetic product which has two visually distinct phases is popular for its eye-catching appearance, and useful because both water-soluble and oil-soluble cosmetic active ingredients can be incorporated.

However, despite the attractive appearance of two-phase or bi-phase cosmetic products, they need to be shake every time before use, and this may be bothersome in actual usage.

Thus, there is a need for a two-phase or bi-phase composition which can have two visually distinct phases which are stable when not being mixed, can be transformed into a single phase composition when being mixed, and can maintain the single phase aspect for a long period of time without mixing, thereby requiring no mixing before the next use.

### DISCLOSURE OF INVENTION

An objective of the present invention is to provide a composition which has two visually distinct phases, can transform into a single phase composition when being mixed, and can maintain the single phase for a long period of time without mixing.

The above objective can be achieved by a two-phase composition comprising:

an oil phase comprising (a) at least one oil, and
an aqueous phase comprising:
(b) at least one crosslinked or non-crosslinked acrylamido-2-methylpropanesulfonic acid (AMPS) homopolymer;
(c) at least one polysaccharide derived from microorganisms; and
(d) water.

The composition according to the present invention is capable of transforming into a single phase composition, and preferably a single phase O/W composition. It is preferable that the single phase composition cannot transform into a two-phase composition within 3 days, preferably within 7 days, and more preferably within 14 days.

The (a) oil may be selected from polar oils, non-polar oils and mixtures thereof, preferably ester oils, ether oils, plant oils, fatty alcohols, hydrocarbons, silicones and mixtures thereof.

The amount of the (a) oil in the composition according to the present invention may be from 0.01% to 40% by weight, preferably from 0.1% to 35% by weight, and more preferably from 1% to 30% by weight, relative to the total weight of the composition.

The amount of the (b) AMPS homopolymer in the composition according to the present invention may be from 0.01% to 5% by weight, preferably from 0.1% to 1% by weight, and more preferably from 0.3% to 0.7% by weight, relative to the total weight of the aqueous phase of the composition.

The (c) polysaccharide derived from microorganisms may be selected from the group consisting of sclerotium gum, xanthan gum and mixtures thereof.

The amount of the (c) polysaccharide derived from microorganisms in the composition according to the present invention may be from 0.001% to 5% by weight, preferably from 0.01% to 1% by weight, and more preferably from 0.05% to 0.5% by weight, relative to the total weight of the aqueous phase of the composition.

The amount of the (d) water in the composition according to the present invention may be from 60% to 95% by weight, preferably from 65% to 90% by weight, and more preferably from 70% to 85% by weight, relative to the total weight of the composition.

The composition according to the present invention may further comprise (e) at least one crosslinked or non-crosslinked copolymer comprising, at least, an AMPS monomer.

The composition according to the present invention may further comprise (f) at least one polysaccharide derived from plants.

The composition according to the present invention may further comprise (g) at least one cosmetic active ingredient, preferably a water-insoluble and oil-insoluble cosmetic active ingredient, and more preferably a water-insoluble and oil-insoluble cosmetic active ingredient derived from microorganisms.

The composition according to the present invention may comprise at least one surfactant in an amount of 1% by weight or less, 0.5% by weight or less, and more preferably 0.3% by weight or less, relative to the total weight of the composition.

The composition according to the present invention may be a cosmetic composition, preferably a skin cosmetic composition, and more preferably a skincare cosmetic composition.

The present invention also relates to a cosmetic process for a keratin substance, preferably skin, comprising applying to the keratin substance the composition according to the present invention.

### BEST MODE FOR CARRYING OUT THE INVENTION

After diligent research, the inventors have discovered that it is possible to provide a composition which has two visually distinct phases, can transform into a single phase composition when being mixed, and can maintain the single phase for a long period of time without mixing.

Thus, one of the aspects of the present invention relates to a two-phase composition comprising:

an oil phase comprising (a) at least one oil, and
an aqueous phase comprising:
(b) at least one crosslinked or non-crosslinked acrylamido-2-methylpropanesulfonic acid (AMPS) homopolymer;
(c) at least one polysaccharide derived from microorganisms; and
(d) water.

The composition according to the present invention can have two visually distinct phases which are stable when not being mixed. However, the composition according to the present invention can be transformed into a single phase composition when being mixed, and can maintain the single phase aspect for a long period of time without mixing. Therefore, it is not necessary to mix the composition according to the present invention every time before use.

The two phases in the composition according to the present invention are an oil phase comprising at least one oil and an aqueous phase comprising water. When mixing the composition according to the present invention, the oil phase can be dispersed in the aqueous phase to form an O/W composition which is visually uniform or forms a single phase. Since the (b) AMPS homopolymer can thicken the aqueous phase to be a gel, the O/W composition can be an O/W gel composition, in particular in the form of an O/W gel emulsion. It is not necessary to use a surfactant or emulsifier to form the O/W gel emulsion.

The composition according to the present invention is stable such that it maintains an oil phase and an aqueous phase when not being mixed firstly. Therefore, the two-phase or bi-phase aspect of the composition according to the present invention can be maintained during transportation or the like of the composition according to the present invention.

The mixing of the composition according to the present invention can be easily performed. For example, the mixing of the composition according to the present invention can be performed by shaking with the hands. After mixing the composition according to the present invention, the composition can form and maintain a single phase for a long period of time without mixing again. Therefore, after the first mixing, it may not be necessary to mix again the composition according to the present invention before use. Of course, additional shaking may be performed, if necessary, but no vigorous shaking is necessary. Therefore, the composition according to the present invention is easy to use and consumer friendly.

The aqueous phase of the composition according to the present invention is stable such that it does not cause phase separation into a gel and water after a long period of time such as more than 1 week.

If the composition according to the present invention further comprises at least one cosmetic active ingredient, the cosmetic active ingredient can be dispersed in the composition. Even if the cosmetic active ingredient is water-insoluble, it can be dispersed in the aqueous phase of the composition according to the present invention as long as the cosmetic active ingredient is hydrophilic. The composition according to the present invention can uniformly disperse the cosmetic active ingredient in the aqueous phase thereof without forming aggregates of the cosmetic active ingredient, and can maintain the uniform dispersion for a long period of time, such as more than 1 week, even at elevated temperature or under temperature change. Thus, the cosmetic active ingredient can be stably dispersed in the composition, preferably the aqueous phase thereof, according to the present invention.

Hereafter, the composition according to the present invention will be described in a detailed manner.

### [Composition]

### (Oil)

The composition according to the present invention comprises (a) at least one oil. If two or more oils are used, they may be the same or different.

The (a) oil forms the oil phase of the composition according to the present invention.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

As examples of plant oils, mention may be made of, for example, meadowfoam oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

As an example of ether oils, mention may be made of dicaprylyl ether.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) (INCI name: Caprylic/Capric Triglyceride) and glyceryl tri(caprate/caprylate/linolenate).

As examples of silicone oils, mention may be made of, for example, linear organopolysiloxanes such as dimethylpolysiloxane (INCI name: Dimethicone), methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:

cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof Mention may also be made of cyclocopolymers of a type such as dimethylsiloxane/methylalkylsiloxane, such as Silicone Volatile® FZ 3109 sold by the company Union Carbide, of formula:

with

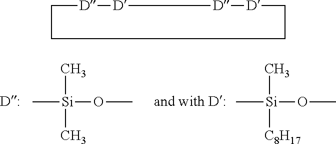

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

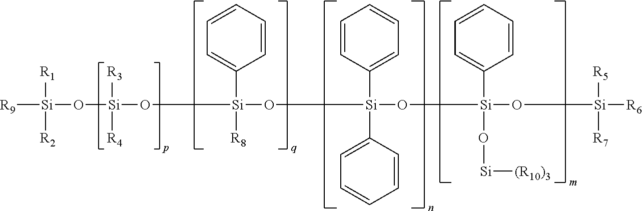

in which

R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and

m, n, p and q are, independently of each other, integers between 0 and 900 inclusive, preferably 0 and 500 inclusive, and more preferably 0 and 100 inclusive, with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, isostearyl alcohol, undecylenyl alcohol, octyldodecanol, hexyldecanol, oleyl alcohol, linoleyl alcohol, palmitoleyl alcohol, arachidonyl alcohol, and mixtures thereof.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, isostearyl alcohol, undecylenyl alcohol, octyldodecanol, hexyldecanol, and mixtures thereof. In one embodiment, octyldodecanol and hexyldecanol can be used as a saturated fatty alcohol.

According to at least one embodiment, the fatty alcohol used in the composition according to the present invention is preferably chosen from octyldodecanol, hexyldecanol and mixtures thereof.

It is preferable that the (a) oil be selected from polar oils, non-polar oils and mixtures thereof, and more preferably ester oils, ether oils, plant oils, fatty alcohols, hydrocarbons, silicones and mixtures thereof.

The amount of the (a) oil in the composition according to the present invention may be 0.01% by weight or more, preferably 0.1% by weight or more, and more preferably 1% by weight or more, relative to the total weight of the composition.

The amount of the (a) oil in the composition according to the present invention may be 40% by weight or less, preferably 35% by weight or less and more preferably 30% by weight or less, relative to the total weight of the composition, with the proviso that the amount of the (a) oil is not zero.

The amount of the (a) oil in the composition according to the present invention may be from 0.01% to 40% by weight, preferably from 0.1% to 35% by weight, and more preferably from 1% to 30% by weight, relative to the total weight of the composition.

### (AMPS Homopolymer)

The composition according to the present invention comprises (b) at least one crosslinked or non-crosslinked acrylamido-2-methylpropanesulfonic acid (AMPS) homopolymer. This polymer is hereafter referred to as AMPS homopolymer. If two or more (b) AMPS homopolymers are used, they may be the same or different.

The (b) AMPS homopolymer can be present in the aqueous phase in the composition according to the present invention. The (b) AMPS homopolymer can function as a hydrophilic thickener which can thicken the aqueous phase of the composition according to the present invention.

The (b) AMPS homopolymer may be preferably totally neutralized or virtually totally neutralized, i.e., at least 90% neutralized.

The (b) AMPS homopolymer may be crosslinked or non-crosslinked.

When the (b) AMPS homopolymer is crosslinked, a crosslinking agent used for crosslinking may be selected from among the polyolefinically unsaturated compounds commonly used for the crosslinking of polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also allylic esters of phosphoric and/or vinylphosphonic acid derivatives, or mixtures of these compounds.

According to one embodiment of the present invention, the crosslinking agent is selected from among methylenebis-acrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The (b) AMPS homopolymer in accordance with the present invention may be water-soluble or water-dispersible.

The term “water-soluble or water-dispersible” means polymers which, when introduced into an aqueous phase at 25° C., at a mass concentration equal to 1%, make it possible to obtain a macroscopically homogeneous and transparent solution, i.e., a solution that has a maximum light transmittance value, at a wavelength equal to 500 nm, through a sample 1 cm thick, of at least 60% and preferably of at least 70%.

The (b) AMPS homopolymer according to the present invention may be prepared by the polymerization of AMPS which may be preferably totally neutralized or virtually totally neutralized, i.e., at least 90% neutralized.

The “homopolymers” according to the present invention are preferably crosslinked and neutralized, and they may be prepared according to a preparation process comprising the following steps:

(a) a monomer such as AMPS in free form is dispersed or dissolved in a solution of tert-butanol or of water and tert-butanol;
(b) the solution or dispersion of monomer obtained in (a) is neutralized with one or more mineral or organic bases, preferably ammonia NH₃, in an amount making it possible to obtain a degree of neutralization of the sulfonic acid functions of the polymer ranging from 90% to 100%;
(c) the crosslinking monomer(s) is(are) added to the solution or dispersion obtained in (b); and
(d) a standard free-radical polymerization is performed in the presence of free-radical initiators at a temperature ranging from 10 to 150° C.; the polymer precipitates in the solution or dispersion based on tert-butanol.

The (b) AMPS homopolymer may have a molar mass ranging from 50,000 g/mol to 10,000,000 g/mol, preferably from 80,000 g/mol to 8,000,000 g/mol and even more preferably from 100,000 g/mol to 7,000,000 g/mol.

Examples of the (b) AMPS homopolymer that may be mentioned include crosslinked or non-crosslinked polymers of sodium acrylamido-2-methylpropanesulfonate, such as the polymer in the commercial product Simulgel 800 (INCI name: Sodium Polyacryloyldimethyltaurate) or Hostacerin AMPS (INCI name: Ammonium Polyacryloyldimethyltaurate).

The amount of the (b) AMPS homopolymer in the composition according to the present invention may be 0.01% by weight or more, preferably 0.1% by weight or more, and more preferably 0.3% by weight or more, relative to the total weight of the aqueous phase of the composition.

The amount of the (b) AMPS homopolymer in the composition according to the present invention may be 5% by weight or less, preferably 1% by weight or less and more preferably 0.7% by weight or less, relative to the total weight of the aqueous phase of the composition, with the proviso that the amount of the (b) AMPS homopolymer is not zero.

The amount of the (b) AMPS homopolymer in the composition according to the present invention may be from 0.01% to 5% by weight, preferably from 0.1% to 1% by weight, and more preferably from 0.3% to 0.7% by weight, relative to the total weight of the aqueous phase of the composition.

The amount of the (b) AMPS homopolymer in the composition according to the present invention may be 0.01% by weight or more, preferably 0.1% by weight or more, and more preferably 0.2% by weight or more, relative to the total weight of the composition.

The amount of the (b) AMPS homopolymer in the composition according to the present invention may be 8% by weight or less, preferably 3% by weight or less and more preferably 0.8% by weight or less, relative to the total weight of the composition, with the proviso that the amount of the (b) AMPS homopolymer is not zero.

The amount of the (b) AMPS homopolymer in the composition according to the present invention may be from 0.01% to 8% by weight, preferably from 0.1% to 3% by weight, and more preferably from 0.2% to 0.8% by weight, relative to the total weight of the composition.

### (Polysaccharide Derived From Microorganisms)

The composition according to the present invention comprises (c) at least one polysaccharide derived from microorganisms. In other words, the (c) polysaccharide is of microorganism origin. A single type of such a polysaccharide may be used, or two or more different types of such polysaccharides may be used in combination.

The (c) polysaccharide derived from microorganisms can be present in the aqueous phase in the composition according to the present invention. The (c) polysaccharide derived from microorganisms can function as a hydrophilic thickener which can thicken the aqueous phase of the composition according to the present invention.

The (c) polysaccharide derived from microorganisms means a polysaccharide produced by microorganisms such as germs or bacteria.

The (c) polysaccharide derived from microorganisms is not a polysaccharide derived from plants. It may be preferable that the (c) polysaccharide derived from microorganisms be not based on cellulose.

As examples of the (c) polysaccharide derived from microorganisms, mention may be made of cardollan, xanthan gum, Jellan gum, dextran, pullulan, sclerotium gum, and mixtures thereof.

It may be preferable that the (c) polysaccharide derived from microorganisms be selected from the group consisting of sclerotium gum, xanthan gum and mixtures thereof.

The amount of the (c) polysaccharide derived from microorganisms in the composition according to the present invention may be 0.001% by weight or more, preferably 0.01% by weight or more, and more preferably 0.05% by weight or more, relative to the total weight of the aqueous phase of the composition.

The amount of the (c) polysaccharide derived from microorganisms in the composition according to the present invention may be 5% by weight or less, preferably 1% by weight or less, and more preferably 0.5% by weight or less, relative to the total weight of the aqueous phase of the composition, with the proviso that the amount of the (c) polysaccharide derived from microorganisms is not zero.

The amount of the (c) polysaccharide derived from microorganisms in the composition according to the present invention may range from 0.001% to 5% by weight, preferably from 0.01% to 1% by weight, and more preferably from 0.05% to 0.5% by weight, relative to the total weight of the aqueous phase of the composition.

The amount of the (c) polysaccharide derived from microorganisms in the composition according to the present invention may be 0.001% by weight or more, preferably 0.01% by weight or more, and more preferably 0.05% by weight or more, relative to the total weight of the composition.

The amount of the (c) polysaccharide derived from microorganisms in the composition according to the present invention may be 3% by weight or less, preferably 0.8% by weight or less, and more preferably 0.3% by weight or less, relative to the total weight of the composition, with the proviso that the amount of the (c) polysaccharide derived from microorganisms is not zero.

The amount of the (c) polysaccharide derived from microorganisms in the composition according to the present invention may range from 0.001% to 3% by weight, preferably from 0.01% to 0.8% by weight, and more preferably from 0.05% to 0.3% by weight, relative to the total weight of the composition.

### (Water)

The composition according to the present invention comprises (d) water.

The (d) water can form an aqueous phase of the composition according to the present invention.

The amount of the (d) water in the composition according to the present invention may be 60% by weight or more, preferably 65% by weight or more, and more preferably 70% by weight or more, relative to the total weight of the composition.

The amount of the (d) water in the composition according to the present invention may be 95% by weight or less, preferably 90% by weight or less and more preferably 85% by weight or less, relative to the total weight of the composition.

The amount of the (d) water in the composition according to the present invention may be from 60% to 95% by weight, preferably from 65% to 90% by weight, and more preferably from 70% to 85% by weight, relative to the total weight of the composition.

### (AMPS Copolymer)

The composition according to the present invention may comprise (e) at least one crosslinked or non-crosslinked copolymer comprising, at least, acrylamido-2-methylpropanesulfonic acid (AMPS) monomer. The polymer is hereafter referred to as AMPS copolymer. If two or more (e) AMPS copolymers are used, they may be the same or different.

The (e) AMPS copolymer can be present in the aqueous phase in the composition according to the present invention. The (e) AMSP copolymer can function as a hydrophilic thickener which can thicken the aqueous phase of the composition according to the present invention.

The (e) AMPS copolymer may be preferably totally neutralized or virtually totally neutralized, i.e., at least 90% neutralized.

The (e) AMPS copolymer may be crosslinked or non-crosslinked.

When the (e) AMPS copolymer is crosslinked, a crosslinking agent used for crosslinking may be selected from among the polyolefinically unsaturated compounds commonly used for the crosslinking of polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also allylic esters of phosphoric and/or vinylphosphonic acid derivatives, or mixtures of these compounds.

According to one embodiment of the present invention, the crosslinking agent is selected from among methylenebis-acrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The (e) AMPS copolymer in accordance with the present invention is water-soluble or water-dispersible.

The term “water-soluble or water-dispersible” means polymers which, when introduced into an aqueous phase at 25° C., at a mass concentration equal to 1%, make it possible to obtain a macroscopically homogeneous and transparent solution, i.e., a solution that has a maximum light transmittance value, at a wavelength equal to 500 nm, through a sample 1 cm thick, of at least 60% and preferably of at least 70%.

The (e) AMPS copolymer may be “copolymers” polymerized from AMPS and from one or more hydrophilic or hydrophobic ethylenically unsaturated monomers and, if they are crosslinked, one or more crosslinking agents such as those defined above.

When the said copolymers are polymerized from hydrophobic ethylenically unsaturated monomers, these monomers do not comprise a fatty chain and are preferably present in small amounts. For the purposes of the present invention, the term “fatty chain” means any hydrocarbon-based chain containing at least 7 carbon atoms.

Thus, the (e) AMPS copolymer may be selected from crosslinked or non-crosslinked copolymers obtained from AMPS and from one or more hydrophilic ethylenically unsaturated monomers or hydrophobic ethylenically unsaturated monomers not containing a fatty chain.

The (e) AMPS copolymer according to the present invention may be prepared by the polymerization of water-soluble ethylenically unsaturated monomers, hydrophobic monomers or mixtures thereof.

The water-soluble comonomers may be ionic or nonionic.

Among the ionic water-soluble comonomers, examples that may be mentioned include the following compounds and the salts thereof:

(meth)acrylic acid,
styrenesulfonic acid,
vinylsulfonic acid and (meth)allylsulfonic acid,
vinylphosphonic acid,
maleic acid,
itaconic acid,
crotonic acid,
the water-soluble vinyl monomers of formula (A) below:

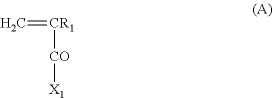

in which:
R₁ is H, —CH₃, —C₂H₅ or —C₃H₇,
X₁ is selected from among:
alkyl ethers of —OR₂ type in which R₂ is a linear or branched, saturated or unsaturated hydrocarbon-based radical containing from 1 to 6 carbon atoms, substituted with at least one sulfonic (—SO₃—) and/or sulfate (—SO₄—) and/or phosphate (—PO₄H₂—) group.
Among the nonionic water-soluble comonomers, examples that may be mentioned include:
(meth)acrylamide,

### N-vinylacetamide and N-methyl-N-vinylacetamide,

### N-vinylformamide and N-methyl-N-vinylformamide,

maleic anhydride,
vinylamine,
N-vinyllactams comprising a cyclic alkyl group containing 4 to 9 carbon atoms, such as n-vinylpyrrolidone, N-butyrolactam and N-vinylcaprolactam,
vinyl alcohol of formula CH₂═CHOH, the water-soluble vinyl monomers of formula (B) below:

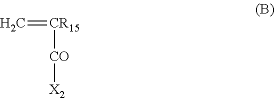

in which:
R₁₅ is H, —CH₃, —C₂H₅ or —C₃H₇;
X₂ is selected from among:
alkyl ethers of —OR₁₆ type in which R₁₆ is a linear or branched, saturated or unsaturated hydrocarbon-based radical containing from 1 to 6 carbons, optionally substituted with a halogen atom (iodine, bromine, chlorine or fluorine); a hydroxyl group (—OH); ether.

Mention is made, for example, of glycidyl (meth)acrylate, hydroxyethyl methacrylate and (meth)acrylates of ethylene glycol, of diethylene glycol or of polyalkylene glycol.

Among the fatty-chain-free hydrophobic comonomers, examples that may be mentioned include:

styrene and its derivatives, such as 4-butylstyrene, α-methylstyrene and vinyltoluene,
vinyl acetate of formula CH₂-CH—OCOCH₃;
vinyl ethers of formula CH₂-CHOR in which R is a linear or branched, saturated or unsaturated hydrocarbon-based radical containing from 1 to 6 carbons;
acrylonitrile,
caprolactone,
vinyl chloride and vinylidene chloride,
silicone derivatives, which provide silicone polymers after polymerization, such as methacryloxypropyltris(trimethylsiloxy)silane and silicone methacrylamides,
the hydrophobic vinyl monomers of formula (C) below:

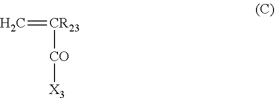

in which:
R₂₃ is H, —CH₃, —C₂H₅ or —C₃H₇;
X₃ is selected from among:
alkyl ethers of —OR₂₄ type in which R₂₄ is a linear or branched, saturated or unsaturated hydrocarbon-based radical containing from 1 to 6 carbon atoms.

Mention is made, for example, of methyl methacrylate, ethyl methacrylate, n-butyl (meth)acrylate, tert-butyl (meth)acrylate, cyclohexyl acrylate, isobornyl acrylate, and 2-ethylhexyl acrylate.

The (e) AMPS copolymer may have a molar mass ranging from 50,000 g/mol to 10,000,000 g/mol, preferably from 80,000 g/mol to 8,000,000 g/mol and even more preferably from 100,000 g/mol to 7,000,000 g/mol.

Examples Of the (e) AMPS copolymer in accordance with the present invention that may be mentioned include:

acrylamide/sodium acrylamido-2-methylpropanesulfonate crosslinked copolymers, such as the copolymer in the commercial product Sepigel 305 (INCI name: Polyacrylamide/C₁₃-C₁₄ Isoparaffin/Laureth-7) or the copolymer in the commercial product sold under the trademark Simulgel 600 (INCI name: Acrylamide/Sodium Acryloyldimethyltaurate/Isohexadecane/P-olysorbate-80) by SEPPIC;
copolymers of AMPS and of vinylpyrrolidone or of vinylformamide, such as the copolymer in the commercial product sold under the name Aristoflex AVC by Clariant (INCI name: Ammonium Acryloyldimethyltaurate/V-P Copolymer) but neutralized with sodium hydroxide or potassium hydroxide;
copolymers of AMPS and of sodium acrylate, for instance AMPS/sodium acrylate copolymer such as the copolymer in the commercial product sold under the name Simulgel EG by SEPPIC (INCI name: Sodium Acrylate/Sodium Acryloyldimethyltaurate Copolymer (and) Isohexadecane (and) Polysorbate-80); and copolymers of AMPS and of hydroxyethyl acrylate, for instance AMPS/hydroxyethyl acrylate copolymer, such as the copolymer in the commercial product sold under the name Sepinov EMT 10 (Hydroxyethyl acrylate/Sodium Acryloyldimethyltaurate copolymer) or Simulgel NS by SEPPIC (INCI name: Hydroxyethyl acrylate/Sodium Acryloyldimethyltaurate copolymer (and) Squalane (and) Polysorbate-60).

Thus, it may be preferable that the (e) AMPS copolymer be selected from the group consisting of:

an acrylamide/sodium acrylamido-2-methylpropanesulfonate (acrylamide/sodium acryloyldimethyltaurate) crosslinked copolymer;
a copolymer of AMPS and of vinylpyrrolidone or of vinylformamide;
a copolymer of AMPS and of sodium acrylate (sodium acrylate/sodium acryloyldimethyltaurate copolymer);
a copolymer of AMPS and of hydroxyethyl acrylate; and
a mixture thereof.

It may be more preferable that the (e) AMPS copolymer be hydroxyethyl acrylate/sodium acryloyldimethyltaurate copolymer.

The amount of the (e) AMPS copolymer in the composition according to the present invention may be 0.01% by weight or more, preferably 0.1% by weight or more, and more preferably 0.3% by weight or more, relative to the total weight of the aqueous phase of the composition.

The amount of the (e) AMPS copolymer in the composition according to the present invention may be 10% by weight or less, preferably 5% by weight or less and more preferably 1% by weight or less, relative to the total weight of the aqueous phase of the composition, with the proviso that the amount of the (e) AMPS copolymer is not zero.

The amount of the (e) AMPS copolymer in the composition according to the present invention may be from 0.01% to 10% by weight, preferably from 0.1% to 5% by weight, and more preferably from 0.3% to 1% by weight, relative to the total weight of the aqueous phase of the composition.

The amount of the (e) AMPS copolymer in the composition according to the present invention may be 0.01% by weight or more, preferably 0.1% by weight or more, and more preferably 0.2% by weight or more, relative to the total weight of the composition.

The amount of the (e) AMPS copolymer in the composition according to the present invention may be 8% by weight or less, preferably 3% by weight or less and more preferably 0.8% by weight or less, relative to the total weight of the composition, with the proviso that the amount of the (e) AMPS copolymer is not zero.

The amount of the (e) AMPS copolymer in the composition according to the present invention may be from 0.01% to 8% by weight, preferably from 0.1% to 3% by weight, and more preferably from 0.2% to 0.8% by weight, relative to the total weight of the composition.

### (Polysaccharide Derived From Plants)

The composition according to the present invention may comprise (f) at least one polysaccharide derived from plants. In other words, the (f) polysaccharide is of plant origin.

A single type of such a polysaccharide may be used, or two or more different types of such polysaccharides may be used in combination.

The (f) polysaccharide derived from plants can be present in the aqueous phase in the composition according to the present invention. The (f) polysaccharide derived from plants can function as a hydrophilic thickener which can thicken the aqueous phase of the composition according to the present invention.

According to the present invention, the term “polysaccharide derived from plants” or “polysaccharide of plant origin” especially means a polysaccharide obtained from the plant kingdom (plants or algae), as opposed to a polysaccharide derived from microorganisms, for example, for xanthan gum, which is produced especially by fermentation of a bacterium, Xanthomonas campestris.

As examples of the (f) polysaccharide derived from plants that may be used according to the present invention, mention may be made especially of:

a) algal extracts, such as alginates, carrageenans and agars, and mixtures thereof. Examples of carrageenans that may be mentioned include Satiagum UTC30® and UTC10® from the company Degussa; an alginate that may be mentioned is the sodium alginate sold under the name Kelcosol® by the company ISP;
b) gums, such as guar gum and nonionic derivatives thereof (hydroxypropyl guar), gum arabic, konjac gum or mannan gum, gum tragacanth, ghatti gum, karaya gum or locust bean gum; examples that may be mentioned include the guar gum sold under the name Jaguar HP105® by the company Rhodia; the mannan and konjac gum® (1% gluconomannan) sold by the company GfN;
c) modified or unmodified starches, such as those obtained, for example, from cereals, for instance wheat, corn or rice, from legumes, for instance blonde peas, from tubers, for instance potato or cassava, and tapioca starches; dextrins, such as corn dextrins; examples that may especially be mentioned include the rice starch Remy DR I® sold by the company Remy; the corn starch B® from the company Roquette; the potato starch modified with 2-chloroethylaminodipropionic acid neutralized with sodium hydroxide, sold under the name Structure Solanace® by the company National Starch; the native tapioca starch powder sold under the name Tapioca Pure® by the company National Starch;
d) dextrins, such as the dextrin extracted from corn under the name Index® from the company National Starch;
e) celluloses and derivatives thereof, in particular alkyl celluloses, hydroxyalkyl celluloses; and alkyl hydroxyalkyl celluloses; mention may be made especially of methyl-celluloses, hydroxyethylcelluloses, ethyl-hydroxyethylcelluloses and carboxymethyl-celluloses. Examples that may be mentioned include stearyl and cetyl hydroxyethylcellulose. Examples of cetyl hydroxyethylcelluloses that may be mentioned include Polysurf 67CS® and Natrosol Plus 330® from Aqualon; and mixtures thereof.

Preferably, the (f) polysaccharide derived from plants may be chosen from an algal extract, a gum and a cellulose derivative, and mixtures thereof. More preferably, agars, locust bean gum, mannan konjac gum, cetyl or stearyl hydroxyethylcelluloses and tapioca starches may be used.

According to a first embodiment, the (f) polysaccharide derived from plants may be an algal extract chosen from alginates, carrageenans and agars, and mixtures thereof. Preferably, alginates or agars, or mixtures thereof, will be used.

According to another embodiment, the (f) polysaccharide derived from plants may be chosen from a gum, such as guar gum, gum arabic, mannan and konjac gum and locust bean (carob) gum, and mixtures thereof.

According to another embodiment, the (f) polysaccharide derived from plants may be a modified or unmodified starch chosen from wheat starch, corn starch, rice starch, potato starch and tapioca starch, and mixtures thereof.

According to another embodiment, the (f) polysaccharide derived from plants may be a dextrin, such as corn dextrin.

According to another embodiment, the (f) polysaccharide derived from plants may be a cellulose derivative. The cellulose derivative may in particular be a (C₁-C₃) hydroxyalkyl cellulose, especially modified with hydrophobic chains, in particular hydrophobic group(s) containing from 8 to 30 carbon atoms. According to one embodiment, the hydrophobic substituent(s) used may be C₈-C₃₀ and preferably C₁₀-C₂₂ alkyl, arylalkyl or alkylaryl groups. Preferably, the hydrophobic substituent(s) according to the present invention may be saturated C₁₀-C₂₂ and preferably C₁₆-C₂₀ alkyl chains, such as cetyl (C₁₆), stearyl (C₁₈) and behenyl (C₂₀) groups. According to one preferred embodiment, the hydrophobic substituent(s) according to the present invention may be cetyl groups. These cellulose derivatives containing hydrophobic substituent(s) according to the present invention may have a viscosity preferably of between 100 and 100 000 mPas and preferably between 200 and 20 000 mPas, measured at 25° C. in a solution containing 1% by weight of a polymer in water, this viscosity being determined conventionally using a viscometer of Brookfield LVT type at 6 rpm with a No. 3 spindle. Among the cellulose derivatives containing hydrophobic substituent(s) that may be used in the compositions of the present invention, mention may preferably be made of the cetyl hydroxyethylcelluloses sold under the names Natrosol Plus Grade 330 CS and Polysurf 67 CS (INCI name: cetyl hydroxyethylcellulose) by the company Aqualon/Hercules.

In one embodiment, the (f) polysaccharide derived from plants may be selected from non-cellulose polysaccharides.

The amount of the (f) polysaccharide derived from plants in the composition according to the present invention may be 0.001% by weight or more, preferably 0.01% by weight or more, and more preferably 0.05% by weight or more, relative to the total weight of the aqueous phase of the composition.

The amount of the (f) polysaccharide derived from plants in the composition according to the present invention may be 5% by weight or less, preferably 1% by weight or less, and more preferably 0.5% by weight or less, relative to the total weight of the aqueous phase of the composition, with the proviso that the amount of the (f) polysaccharide derived from plants is not zero.

The amount of the (f) polysaccharide derived from plants in the composition according to the present invention may range from 0.001% to 5% by weight, preferably from 0.01% to 1% by weight, and more preferably from 0.05% to 0.5% by weight, relative to the total weight of the aqueous phase of the composition.

The amount of the (f) polysaccharide derived from plants in the composition according to the present invention may be 0.001% by weight or more, preferably 0.01% by weight or more, and more preferably 0.05% by weight or more, relative to the total weight of the composition.

The amount of the (f) polysaccharide derived from plants in the composition according to the present invention may be 3% by weight or less, preferably 0.8% by weight or less, and more preferably 0.3% by weight or less, relative to the total weight of the composition, with the proviso that the amount of the (f) polysaccharide derived from plants is not zero.

The amount of the (f) polysaccharide derived from plants in the composition according to the present invention may range from 0.001% to 3% by weight, preferably from 0.01% to 0.8% by weight, and more preferably from 0.05% to 0.3% by weight, relative to the total weight of the composition.

### (Cosmetic Active Ingredient)

The composition according to the present invention may comprise (g) at least one cosmetic active ingredient. A single type of the (g) cosmetic active ingredient, or two or more different types of the (g) cosmetic active ingredient may be used in combination.

The (g) cosmetic active ingredient may preferably be a water-insoluble and oil-insoluble cosmetic active ingredient, and more preferably water-insoluble and oil-insoluble cosmetic active ingredient derived from microorganisms.

In an embodiment of the present invention, the (g) cosmetic active ingredient may be a lysate of microorganisms, for example a lysate of the genus Bifidobacterium.

A lysate commonly denotes a material obtained at the end of the destruction or dissolution of biological cells by means of a phenomenon termed cell lysis, thus causing release of the intracellular biological constituents naturally contained in the cells of the microorganism under consideration.

For the purpose of the present invention, the term “lysate” is used without distinction to denote the entire lysate obtained by lysis of the microorganism concerned, or only a fraction thereof.

Thus, it is preferable that the (g) cosmetic active ingredient be a lysate of a Bifidobacterium species and/or a fraction thereof.

The lysate used is therefore entirely or partly formed from the intracellular biological constituents and from the constituents of the cell walls and membranes.

More specifically, it contains the cytoplasmic cell fraction containing the enzymes such as lactic acid dehydrogenase, phosphatases, phosphoketolases and transaldolases, and the metabolites. By way of illustration, the cell wall constituents are in particular peptidoglycan, murein or mucopeptide and teichoic acid, and the cell membrane constituents are composed of glycerophospholipid.

This cell lysis may be accomplished by means of various technologies, such as, for example, osmotic shock, heat shock, by ultrasound, or else under mechanical stress of the centrifugation type.

More particularly, this lysate can be obtained according to the technology described in U.S. Pat. No. 4,464,362, and in particular according to the following protocol.

A microorganism of Bifidobacterium type under consideration is cultured anaerobically in a suitable culture medium, for example according to the conditions described in documents U.S. Pat. No. 4,464,362 and EP 0 043 128. When the stationary phase of development has been reached, the culture medium can be inactivated by pasteurization, for example at a temperature of from 60 to 65° C. for 30 minutes. The microorganisms are then recovered by means of a conventional separation technique, for example membrane filtration, centrifuged, and resuspended in a sterile solution of NaCl at a physiological concentration. The lysate can be obtained by disintegration of such a medium with ultrasound, in order to release therefrom the cytoplasmic fractions, the cell wall fragments and the metabolism-derived products. Next, all the components in their natural distribution are subsequently stabilized in a weakly acidic aqueous solution.

A lysate having a concentration on the order of from 0.1% to 50%, in particular from 1% to 20%, and especially approximately 5% by weight of active material(s), relative to the total weight thereof, is thus generally obtained.

The lysate may be used in various forms, in the form of a solution or in pulverulent form.

A microorganism most particularly suitable for the present invention, belongs to the genus Bifidobacterium and is preferably chosen from the species: Bifidobacterium longum, Bifidobacterium bifidum, Bifidobacterium breve, Bifidobacterium animalis, Bifidobacterium lactis, Bifidobacterium infantis, Bifidobacterium adolescentis or Bifidobacterium pseudocatenulatum, and mixtures thereof.

The species Bifidobacterium longum is most particularly suitable for the present invention.

The lysate may advantageously be the lysate registered under the INCI name: Bifida Ferment Lysate, under the EINECS name: Bifidobacterium longum, under EINECS No.: 306-168-4 and under CAS No.: 96507-89-0.

The product sold under the name Repair Complex CLR® by the company K. Richter GmbH and which is formed from an inactivated lysate of the species Bifidobacterium longum, falls within the context of the present invention.

The amount of the (g) cosmetic active ingredient in the composition according to the present invention may be 0.1% by weight or more, preferably 1% by weight or more, and more preferably 5% by weight or more, relative to the total weight of the aqueous phase of the composition.

The amount of the (g) cosmetic active ingredient in the composition according to the present invention may be 30% by weight or less, preferably 25% by weight or less, and more preferably 20% by weight or less, relative to the total weight of the aqueous phase of the composition, with the proviso that the amount of the (g) cosmetic active ingredient is not zero.

The amount of the (g) cosmetic active ingredient in the composition according to the present invention may range from 0.1% to 30% by weight, preferably from 1% to 25% by weight, and more preferably from 5% to 20% by weight, relative to the total weight of the aqueous phase of the composition.

The amount of the (g) cosmetic active ingredient in the composition according to the present invention may be 0.1% by weight or more, preferably 1% by weight or more, and more preferably 5% by weight or more, relative to the total weight of the composition.

The amount of (g) cosmetic active ingredient in the composition according to the present invention may be 25% by weight or less, preferably 20% by weight or less, and more preferably 15% by weight or less, relative to the total weight of the composition, with the proviso that the amount of the (g) cosmetic active ingredient is not zero.

The amount of the (g) cosmetic active ingredient in the composition according to the present invention may range from 0.1% to 25% by weight, preferably from 1% to 20% by weight, and more preferably from 5% to 15% by weight, relative to the total weight of the composition.

### (Surfactant)

The composition according to the present invention may comprise at least one surfactant. If two or more surfactants are used, they may be the same or different.

However, it may be preferable that the amount of the surfactant is small.

The amount of the surfactant may be 1% by weight or less, preferably 0.5% by weight or less, and more preferably 0.3% by weight or less, relative to the total weight of the composition according to the present invention. It is in particular preferable that the composition according to the present invention comprise no surfactant.

### (Other Ingredients)

The composition according to the present invention may also include at least one optional or additional ingredient.

The amount of the optional or additional ingredient(s) is not limited, but may be from 0.01% to 30% by weight, preferably from 0.1% to 20% by weight, and more preferably from 1% to 10% by weight, relative to the total weight of the composition according to the present invention.

The optional or additional ingredient(s) may be selected from the group consisting of anionic, cationic, nonionic, or amphoteric polymers; organic or inorganic UV filters; peptides and derivatives thereof; protein hydrolyzates; swelling agents and penetrating agents; agents for combating hair loss; anti-dandruff agents; hydrophobic thickeners; suspending agents; sequestering agents; opacifying agents; dyes; vitamins or provitamins; fragrances; preservatives, co-preservatives, stabilizers; and mixtures thereof.

The composition according to the present invention may include one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ethers, and butylene glycol monomethyl, monoethyl, and monobutyl ethers.

The organic solvent(s) may then be present in a concentration of from 0.01% to 20% by weight, preferably from 0.1% to 15% by weight, and more preferably from 1% to 10% by weight, relative to the total weight of the composition.

The pH of the composition according to the present invention may be controlled. The pH may be, for example, from 3 to 11, preferably from 3.5 to 9, and more preferably from 4 to 7. The pH may be adjusted to the desired value using at least one acidifying agent and/or at least one basifying agent.

The acidifying agents can be, for example, mineral or organic acids, for instance hydrochloric acid, orthophosphoric acid, carboxylic acid, for instance tartaric acid, citric acid, lactic acid, or sulphonic acids.

The basifying agent can be, for example, ammonium hydroxide, alkali metal hydroxide, alkali earth metal hydroxide, alkali metal carbonates, alkanolamines such as mono-, di-, and triethanolamines, and also their derivatives, preferably sodium or potassium hydroxide and compounds of the formula below:

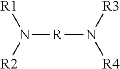

wherein

R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine and derivatives thereof. Arginine, urea, and monoethanolamine may be preferable.

The acidifying or basifying agent may be present in an amount ranging from less than 5% by weight, preferably from 1% by weight or less, and more preferably from 0.1% by weight or less, relative to the total weight of the composition.

### (Form)

The composition according to the present invention can have two visually distinct phases. One of the two phases is an oil phase, and the other is an aqueous phase.

The composition according to the present invention can be transformed into a single phase composition when being mixed. The composition according to the present invention after mixing can be of the O/W type, preferably in the form of an O/W gel, and more preferably an O/W gel emulsion.

The O/W architecture or structure, which comprises oil phases dispersed in an aqueous phase, has an external aqueous phase, and therefore products based on the O/W architecture or structure are more pleasant to use because of the feeling of immediate freshness that they can provide.

When the composition according to the present invention is transformed into the O/W type, it comprises oil phases dispersed in a continuous aqueous phase. The dispersed oil phases can be recognized as oil droplets in the aqueous phase.

It may be preferable that the composition according to the present invention of the O/W type be in the form of a fine emulsion, more preferably a nano- or micro-emulsion, and even more preferably a nano-emulsion.

### [Preparation]

The composition according to the present invention can be prepared by mixing the ingredients (a) to (d) as well as optional or additional ingredient(s).

For example, the composition according to the present invention can be prepared by the process comprising:

(i) mixing
(b) at least one AMPS homopolymer;
(c) at least one polysaccharide derived from microorganisms; and
(d) water
to form an aqueous phase,
   and
   (ii) adding (a) at least one oil to the aqueous phase
to form an oil phase on the aqueous phase.

The mixing step can be performed by any conventional means.

It is preferable that the addition of the (a) oil to the aqueous phase be performed gently such that the (a) oil is not dispersed in the aqueous phase.

### [Use and Process]

When using the composition according to the present invention, it will be mixed by, for example, shaking with the hands (hand-shaking). After mixing the composition according to the present invention, the composition can form a single phase. Since the single phase can be maintained for a long period of time after the first use, a user of the composition according to the present invention may not need to mix again it before the second use.

The composition according to the present invention may preferably be used as a cosmetic composition, more preferably a skin cosmetic composition, and even more preferably a skincare cosmetic composition.

The composition according to the present invention can provide cosmetic effects such as a fresh sensation, good texture, and soft skin finish.

The skin here encompasses facial skin, neck skin, and the scalp. The composition according to the present invention may also be used for mucosae such as the lips, and the like.

The composition according to the present invention can be used as it is (as a topical product), or can be used by being impregnated into a porous substrate such as a non-woven fabric preferably made from cellulose fibers to prepare a cosmetic product such as a cosmetic mask.

In particular, the composition according to the present invention may be intended for application onto a keratin substance such as the skin or lips, preferably the skin. Thus, the composition according to the present invention can be used for a cosmetic process for the skin or lips, preferably the skin.

It is preferable that the composition according to the present invention be used for skincare, not for skin makeup. In other words, it is preferable that the composition according to the present invention be used for skincare products such as a lotion and a cream, not for skin makeup products such as a foundation. Preferably the composition according to the present invention does not comprise iron oxide, or comprises iron oxide in an amount of 0.5% by weight or less, more preferably 0.2% by weight or less, even more preferably 0.1% by weight or less, relative to the total weight of the composition according to the present invention.

The cosmetic process for a keratin substance such as the skin, according to the present invention may comprise, at least, the step of applying onto the keratin substance the composition according to the present invention.

The present invention also relates to the use of

(b) at least one crosslinked or non-crosslinked acrylamido-2-methylpropanesulfonic acid (AMPS) homopolymer, and
(c) at least one polysaccharide derived from microorganisms; and in a composition comprising (d) water, in order to provide an aqueous phase which can disperse oil phases comprising (a) at least one oil for a long period of time even after mixing the aqueous phases and the oil phases.

In addition, the present invention also relates to the use of

(b) at least one crosslinked or non-crosslinked acrylamido-2-methylpropanesulfonic acid (AMPS) homopolymer, and
(c) at least one polysaccharide derived from microorganisms; and in a composition comprising (d) water, in order to stably disperse (g) at least one cosmetic active ingredient in an aqueous phase comprising (d) water. The composition may further comprise an oil phase or oil phases comprising (a) at least one oil.

### EXAMPLES

The present invention will be described in a more detailed manner by way of examples. However, these examples should not be construed as limiting the scope of the present invention. The examples below are presented as non-limiting illustrations in the field of the present invention.

### Examples 1-7 and Comparative Examples 1-5

The following compositions according to Examples 1-7 and Comparative Examples 1-5 shown in Tables 1 and 2, were prepared by mixing the ingredients shown in Tables 1 and 2 as follows. The pH of the composition was adjusted to be 4.9 by adding sodium hydroxide. The numerical values for the amounts of the ingredients shown in Tables 1 and 2 are all based on “% by weight” as active raw materials.

TABLE 1

| | Ex. 1 | Ex. 2 | Ex. 3 | Ex. 4 | Ex. 5 | Ex. 6 | Ex. 7 |
|---|---|---|---|---|---|---|---|
| Water | qsp 100 | qsp 100 | qsp 100 | qsp 100 | qsp 100 | qsp 100 | qsp 100 |
| Glycerin | 6.67 | 6.67 | 6.67 | 6.67 | 6.67 | 6.67 | 6.67 |
| Pentylene Glycol | 4.00 | 4.00 | 4.00 | 4.00 | 4.00 | 4.00 | 4.00 |
| Mannose | 0.67 | 0.67 | 0.67 | 0.67 | 0.67 | 0.67 | 0.67 |
| Chlorphenesin | 0.27 | 0.27 | 0.27 | 0.27 | 0.27 | 0.27 | 0.27 |
| Salicylic Acid | 0.26 | 0.26 | 0.26 | 0.26 | 0.26 | 0.26 | 0.26 |
| Propylene Glycol | 6.00 | 6.00 | 6.00 | 6.00 | 6.00 | 6.00 | 6.00 |
| Trisodium Ethylenediamine Disuccinate | 0.05 | 0.05 | 0.05 | 0.05 | 0.05 | 0.05 | 0.05 |
| NOMCORT CG | 0.05 | 0.05 | 0.05 | 0.05 | 0.04 | 0.05 | — |
| Xanthan Gum | — | — | — | — | — | — | 0.05 |
| Ceratonia Siliqua (Carob) Gum | — | — | — | — | — | — | — |
| Bifida Ferment Lysate | 13.15 | 13.15 | 13.15 | 13.15 | 13.15 | 13.15 | 13.15 |
| Ammonium Polyacryloyldimethyl Laurate | 0.30 | 0.35 | 0.50 | 0.70 | 0.35 | 0.35 | 0.35 |
| Hydroxyethyl Acrylate/Sodium | — | — | — | — | — | 0.35 | — |
| Acryloyldimethyl Taurate Copolymer | | | | | | | |
| Acrylates/C10-30 Alkyl Acrylate Crosspolymer | — | — | — | — | — | — | — |
| Hydroxyethylpiperazine Ethane Sulfonic Acid | 1.34 | 1.34 | 1.34 | 1.34 | 1.34 | 1.34 | 1.34 |
| Alpha-Glucan Oligosaccharide | 0.28 | 0.28 | 0.28 | 0.28 | 0.28 | 0.28 | 0.28 |
| Polymnia Sonchifolia Root Juice | 0.08 | 0.08 | 0.08 | 0.08 | 0.08 | 0.08 | 0.08 |
| Lactobacillus | 0.004 | 0.004 | 0.004 | 0.004 | 0.004 | 0.004 | 0.004 |
| Yeast Extract | 0.06 | 0.06 | 0.06 | 0.06 | 0.06 | 0.06 | 0.06 |
| Sodium Hydroxide | qs | qs | qs | qs | qs | qs | qs |
| | pH4.9 | pH4.9 | pH4.9 | pH4.9 | pH4.9 | pH4.9 | pH4.9 |
| Stability (10 cycles) | Stable | Stable | Stable | Stable | Stable | Stable | Stable |
| Stability (50° C. 2 weeks) | Stable | Stable | Stable | Stable | Stable | Stable | Stable |
| Stability (after shaking with oil) | 3 | 4 | 4 | 4 | 3 | 4 | 3 |

TABLE 2

| | Comp. | Comp. | Comp. | Comp. | Comp. |
|---|---|---|---|---|---|
| | Ex. 1 | Ex. 2 | Ex. 3 | Ex. 4 | Ex. 5 |
| Water | qsp 100 | qsp 100 | qsp 100 | qsp 100 | qsp 100 |
| Glycerin | 6.67 | 6.67 | 6.67 | 6.67 | 6.67 |
| Pentylene Glycol | 4.00 | 4.00 | 4.00 | 4.00 | 4.00 |
| Mannose | 0.67 | 0.67 | 0.67 | 0.67 | 0.67 |
| Chlorphenesin | 0.27 | 0.27 | 0.27 | 0.27 | 0.27 |
| Salicylic Acid | 0.26 | 0.26 | 0.26 | 0.26 | 0.26 |
| Propylene Glycol | 6.00 | 6.00 | 6.00 | 6.00 | 6.00 |
| Trisodium Ethylenediamine Disuccinate | 0.05 | 0.05 | 0.05 | 0.05 | 0.05 |
| NOMCORT CG | — | 0.05 | 0.05 | 0.05 | — |
| Xanthan Gum | — | — | — | — | — |
| Ceratonia Siliqua (Carob) Gum | 0.05 | — | — | — | — |
| Bifida Ferment Lysate | 13.15 | 13.15 | 13.15 | 13.15 | 13.15 |
| Ammonium Polyacryloyldimethyl Taurate | 0.35 | — | — | — | 035 |
| Hydroxyethyl Acrylate/Sodium | — | 0.70 | 0.35 | — | 035 |
| Acryloyldimethyl Taurate Copolymer | | | | | |
| Acrylates/C10-30 Alkyl Acrylate Crosspolymer | — | — | — | 0.35 | — |
| Hydroxyethylpiperazine Ethane Sulfonic Acid | 1.34 | 1.34 | 1.34 | 1.34 | 1.34 |
| Alpha-Glucan Oligosaccharide | 0.28 | 0.28 | 0.28 | 0.28 | 0.28 |
| Pofymnia Sortchifolia Root Juice | 0.08 | 0.08 | 0.08 | 0.08 | 0.08 |
| Lactobacillus | 0.004 | 0.004 | 0.004 | 0.004 | 0.004 |
| Yeast Extract | 0.06 | 0.06 | 0.06 | 0.06 | 0.06 |
| Sodium Hydroxide | qs pH4.9 | qs pH4.9 | qs pH4.9 | qs pH4.9 | qs pH4.9 |
| Stability (10 cycles) | Stable | Stable | Unstable | Stable | Stable |
| Stability (50° C. 2 weeks) | Stable | Stable | Unstable | Stable | Stable |
| Stability (after shaking with oil) | 2 | 1 | 1 | 2 | 1 |

NOMCORT CG: (Xanthan Gum (and) Ceratonia Siliqua (Carob) Gum) by Nisshin Oillio

### [Evaluation 1]

The compositions according to Examples 1-7 and Comparative Examples 1-5 were evaluated as follows.

(Stability (10 cycles))

38 g each of the compositions according to Examples 1-7 and Comparative Examples 1-5 were charged into a 50-ml vial, and the vial was put into an oven for 10 days. In the oven, the temperature of the vial was controlled by being maintained at a constant temperature of 20° C. for 6 hours, decreased to −20° C. for 6 hours, maintained at a constant temperature of −20° C., and increased to 20° C. for 6 hours. The above temperature cycle took 1 day. The above temperature cycle was repeated 10 times for 10 days. After 10 days, the aspect of each composition was visually observed and evaluated in accordance with the following criteria.

Stable: No change in aspect before and after the stability test
Unstable: Small aggregates were observed after the stability test

The small aggregates were derived from Bifida Ferment Lysate.

The results are shown in Tables 1 and 2.

(Stability (50° C. 2 weeks))

38 g each of the compositions according to Examples 1-7 and Comparative Examples 1-5 were charged into a 50-ml vial, and the vial was put into an oven for 14 days. In the oven, the temperature of the vial was controlled by being maintained at a constant temperature of 50° C. for 14 days. After 14 days, the aspect of each composition was visually observed and evaluated in accordance with the following criteria.

Stable: No change in aspect before and after the stability test
Unstable: Uneven in aspect after the stability test

The unstable compositions were not homogeneous and were partly jellified with a liquid.

The results are shown in Tables 1 and 2.

(Stability (after shaking with oil))

7.5 g each of the compositions according to Examples 1-7 and Comparative Examples 1-5 and 2.5 g of oil were charged into a 20-ml vial. The formulation of the oil is shown in Table 3. A two-phase formulation, in which one phase (aqueous phase) is composed of the composition according to each of the compositions according to Examples 1-7 and Comparative Examples 1-5, and the other phase (oil phase) is composed of the oil, was formed in the vial. The two-phase formulation corresponds to the composition according to the present invention. The vial was shaken 60 times vigorously by hand-shaking to mix the composition and oil. After 72 hours, the aspect of each vial was visually observed and evaluated in accordance with the following criteria.

4: A single phase is observed (oil is completely emulsified)
3: A single phase is observed, but very slight amount of water can be observed when the bottle is tilted. A single phase can be formed after re-shaking 1 or 2 times by hand.
2: A phase separation (a small amount of water is present at the bottom) is observed, but a single phase can be formed by re-shaking more than a few times, e.g., more than 5 times, again
1: Two phases (with an emulsified opaque upper phase and a translucent lower aqueous phase) are observed

TABLE 3

| | Wt % |
|---|---|
| Isopropyl Myristate | 3.40 |
| Dicaprylyl Ether | 31.5 |
| Octyl-2-Dodecanol | 45 |
| Helianthus Annuus (Sunflower) Seed Oil Unsaponifiables | 8 |
| Limnanthesis Alba (Meadowfoam) Seed Oil | 8 |
| Tocopherol | 2 |
| Tocopheryl Acetate | 2 |
| Fragrance | 0.1 |

It can be understood from the above results regarding Examples 1-7 that the compositions according to the present invention can form a single phase by being mixed, and can maintain the single phase for a long period of time.

In view of the results regarding Example 6 and Comparative Example 5, it can be understood that the absence of a polysaccharide derived from microorganisms will cause deterioration in the stability of the single phase composition obtained by mixing with oil.

In view of the results regarding Comparative Examples 2-4, it can be understood that the absence of AMPS homopolymer will also cause deterioration in the stability of the single phase composition obtained by mixing with oil.

### [Evaluation 2]

10 g each of the composition according to Example 3 as an aqueous phase and various amounts of oil as an oil phase were charged into a 20-ml vial. The formulation of the oil is shown in Table 3. A two-phase formulation, in which one phase (aqueous phase) is composed of the composition according to Example 3, and the other phase (oil phase) is composed of the oil, was formed in the vial. The two-phase formulation corresponds to the composition according to the present invention. The weight ratio of the oil phase:the aqueous phase is shown in Table 4. The vial was shaken 60 times vigorously by hand-shaking to mix the composition and oil. After 72 hours; the aspect of each vial was visually observed and evaluated in accordance with the following criteria. The results of the observation are shown in Table 4.

4: A single phase is observed (oil is completely emulsified)
3: A very slight phase separation (a slight amount of oil is present at the top) is observed

TABLE 4

| | | | | | |
|---|---|---|---|---|---|
| Oil Phase:Aqueous Phase | 20:80 | 25:75 | 30:70 | 35:65 | 40:60 |
| (Weight Ratio) | | | | | |
| Aspect | 4 | 4 | 4 | 4 | 3 |

It can be understood from the above results that the composition according to the present invention can form a single phase by being mixed, and can maintain the single phase for a long period of time, in particular, if the amount of oil is 40% by weight or less, preferably 35% by weight or less, and more preferably 30% by weight or less, relative to the total weight of the composition.

### [Evaluation 3]

7.5 g each of the composition according to Example 3 as an aqueous phase and 2.5 g of a single type of oil as an oil phase were charged into a 20-ml vial. The type of the oil is shown in Table 5. A two-phase formulation, in which one phase (aqueous phase) is composed of the composition according to Example 3, and the other phase (oil phase) is composed of the oil, was formed in the vial. The two-phase formulation corresponds to the composition according to the present invention. The vial was shaken 60 times vigorously by hand-shaking to mix the composition and oil. After 72 hours at 45° C., the aspect of each vial was visually observed and evaluated in accordance with the following criteria. The results of the observation are shown in Table 5.

4: A single phase is observed (oil is completely emulsified)
3: A very slight phase separation (a slight amount of oil is present at the top) is observed

TABLE 5

| Isopropyl Myristate | Aspect |
|---|---|
| Dicaprylyl Ether | 4 |
| Octyl-2-Dodecanol | 4 |
| Meadowfoam Oil | 4 |
| Dimethicone | 4 |
| Cyclohexasiloxane | 4 |
| Mineral Oil | 4 |
| Caprylic/Capric Triglyceride | 4 |
| Squalane | 4 |

It can be understood from the above results that the composition according to the present invention can form a single phase by being mixed, and can maintain the single phase even if various types of oil are used.

### [Evaluation 3]

7.5 g each of the composition as shown in Table 6 as an aqueous phase and 2.5 g of oil as an oil phase was charged into a 20 ml vial. The formulation of the oil is shown in Table 3. A two-phase formulation, in which one phase (aqueous phase) is composed of the composition shown in Table 6, and the other phase (oil phase) is composed of the oil, was formed in the vial. The two-phase formulation corresponds to the composition according to the present invention. The vial was shaken 60 times vigorously by hand-shaking to mix the composition and oil.

Using a pipette, 2 drops of the mixture thus obtained were applied on forearm of 7 panelists. The skin affinity and skin softness after drying were evaluated in accordance with the following score criteria, and the average score was calculated. The results of the evaluation is shown in Table 6. Sample A was set as a benchmark to compare, and therefore, the score was set as 3.

### (Skin Affinity)

| | |
|---|---|
| 5 | Very good skin affinity with good penetration feeling |
| 4 | Good skin affinity with penetration feeling |
| 3 | Neutral (Same as a benchmark) |
| 2 | Not good skin affinity with less penetration feeling |
| 1 | Bad skin affinity with very less penetration feeling |

### (Skin Softness After Drying)

| | |
|---|---|
| 5 | Very soft skin finish |
| 4 | Soft skin finish |
| 3 | Neutral (same as benchmark) |
| 2 | Rigid skin finish |
| 1 | Very rigid skin finish |

TABLE 6

| | Sample A | Sample B | Sample C | Sample D |
|---|---|---|---|---|
| Oil Phase | Oil (cf. Table 3) | | | |
| Aqueous Phase | Ex. 2 | Ex. 3 | Ex. 4 | Ex. 6 |
| Skin Affinity | 3.00 | 3.14 | 3.29 | 3.29 |
| Skin Softness | 3.00 | 3.00 | 3.00 | 3.86 |
| after Drying | | | | |

It can be understood from the above results that the composition according to the present invention can provide cosmetic effects for a keratin substance such as skin. For example, the composition according to the present invention can have good affinity to skin, and can provide skin with good penetration feeling. Also, the composition according to the present invention can provide skin with softness.

1. A two phase composition comprising
an oil phase comprising (a) at least one oil, and
an aqueous phase comprising:
(b) at least one crosslinked or non-crosslinked acrylamido-2-methylpropanesulfonic acid (AMPS) homopolymer;
(c) at least one polysaccharide derived from microorganisms; and
(d) water.

2. The composition according to claim 1,
wherein the composition is capable of transforming into a single phase composition.

3. The composition according to claim 2,
wherein the single phase composition is not capable of transforming into a two phase composition within 3 days.

4. The composition according to claim 1, wherein the (a) oil is selected from polar oils, non-polar oils and mixtures thereof.

5. The composition according to claim 1, wherein the amount of the (a) oil in the composition is from 0.01% to 40% by weight, relative to the total weight of the composition.

6. The composition according to claim 1, wherein the amount of the (b) AMPS homopolymer in the composition is from 0.01% to 5% by weight, relative to the total weight of the aqueous phase of the composition.

7. The composition according to claim 1, wherein the (c) polysaccharide derived from microorganisms is selected from the group consisting of sclerotium gum, xanthan gum and mixtures thereof.

8. The composition according to claim 1, wherein the amount of the (c) polysaccharide derived from microorganisms in the composition is from 0.001% to 5% by weight, relative to the total weight of the aqueous phase of the composition.

9. The composition according to claim 1, wherein the amount of the (d) water in the composition is from 60% to 95% by weight, relative to the total weight of the composition.

10. The composition according to claim 1, wherein the composition further comprises (e) at least one crosslinked or non-crosslinked copolymer comprising, at least, an AMPS monomer.

11. The composition according to claim 1, wherein the composition further comprises (f) at least one polysaccharide derived from plants.

12. The composition according to claim 1, wherein the composition further comprises (g) at least one cosmetic active ingredient.

13. The composition according to claim 1, wherein the composition comprises at least one surfactant in an amount of 1% by weight or less, relative to the total weight of the composition.

14. The composition according to claim 1, wherein the composition is a cosmetic composition.

15. A cosmetic process for a keratin substance, comprising applying to the keratin substance the composition according to claim 1.
